(19) United States
(12) Patent Application Publication
(10) Pub. No.: US 2025/0079009 A1
(43) Pub. Date: Mar. 6, 2025

(54) METHODS AND SYSTEMS FOR CHROMATOGRAPHIC DETERMINATION OF A METABOLOME

(71) Applicant: Park City Venture Partners, LLC, Park City, UT (US)

(72) Inventors: Martin MARTINOV, Park City, UT (US); Dejan NENOV, Boise, ID (US)

(21) Appl. No.: 18/824,159

(22) Filed: Sep. 4, 2024

Related U.S. Application Data
(63) Continuation of application No. PCT/US2024/045079, filed on Sep. 4, 2024.
(60) Provisional application No. 63/580,746, filed on Sep. 6, 2023.

(51) Int. Cl. G16H 50/20; G01N 30/72; G01N 30/86; G16C 20/70; G16H 10/60

(52) U.S. Cl. CPC G16H 50/20; G01N 30/8675; G16C 20/70; G16H 10/60; G01N 30/72

(57) ABSTRACT

In an aspect, the present disclosure provides a method of determining a metabolic profile of a subject, comprising: (a) obtaining chromatography data obtained from a biological sample of said subject; (b) processing, using a machine-learning (ML) algorithm, a set of input features of said chromatography data to generate output data, wherein said set of input features do not comprise a presence or a quantity of a metabolite of said biological sample; and (c) determining said metabolic profile based at least in part on said output data.

### CROSS-REFERENCE

This application claims the benefit of U.S. Provisional Application No. 63/580,746, filed Sep. 6, 2023, which is incorporated by reference herein in its entirety.

### BACKGROUND

Many diseases can be linked to or impact the metabolism of the person who has the disease. Non-invasive, early disease determination can provide improvements to the outcomes of patients.

### SUMMARY

In an aspect, the present disclosure provides a method for determining a metabolic profile of a subject, comprising: (a) obtaining chromatography data obtained from a biological sample of the subject; (b) processing, using a machine-learning (ML) algorithm, a set of input features of the chromatography data to generate output data, wherein the set of input features do not comprise a presence or a quantity of a metabolite of the biological sample; and (c) determining the metabolic profile based at least in part on the output data.

In some embodiments, the method further comprises, (d) processing the metabolic profile to determine a presence or an absence of a disease state. In some embodiments, the processing in (d) further comprises processing a characteristic of the subject. In some embodiments, the characteristic is selected from the group consisting of previous tumor, demographics, clinical characteristic, demographic characteristic, and phenotypic characteristic. In some embodiments, the disease state is selected from the group consisting of an oncological disease, an infectious disease, a chronic disease, a nutritional deficiency, an environmental disease, an autoimmune disorder, and a genetic disease. In some embodiments, the disease state comprises a plurality of disease states, and wherein the method further comprises processing the metabolic profile to determine a presence or absence of each of the plurality of disease states. In some embodiments, the plurality of disease states comprises at least about 5 disease states. In some embodiments, the plurality of disease states comprises at least about 50 disease states. In some embodiments, the presence or the absence of the disease state is determined at an accuracy of at least about 85%. In some embodiments, the presence or the absence of the disease state is determined using a single sample from the subject. In some embodiments, the method further comprises processing the metabolic profile to determine a presence or an absence of use of a compound by the subject. In some embodiments, the biological sample is a urine sample. In some embodiments, the chromatography data is derived from a gas chromatography system. In some embodiments, the chromatography data is derived from a liquid chromatography system. In some embodiments, the set of input features do not comprise a presence or a quantity of an analyte of the biological sample. In some embodiments, the ML algorithm comprises a fuzzy decision network. In some embodiments, the method further comprises repeating (a)-(c) for a plurality of biological samples of a plurality of subjects to generate a plurality of metabolic profiles. In some embodiments, the method further comprises analyzing the plurality of metabolic profiles to determine a differential feature of the plurality of metabolic profiles. In some embodiments, the method further comprises, prior to (a), performing chromatography on the biological sample to generate the chromatography data. In some embodiments, the chromatography does not comprise derivatization of the biological sample. In some embodiments, the biological sample is an unpreserved biological sample. In some embodiments, the unpreserved biological sample is a raw biological sample. In some embodiments, the method further comprises, subsequent to (a), processing the sample with a mass spectrometer. In some embodiments, the method further comprises, determining the presence or the quantity of the metabolite. In some embodiments, the method further comprises determining the metabolic profile based further on the presence or the quantity of the metabolite. In some embodiments, the output data comprises the presence or the quantity of the metabolite. In some embodiments, the chromatography data comprises a first gas chromatography data and a second liquid chromatography data. In some embodiments, the set of input features further comprises additional data. In some embodiments, the additional data comprises additional data selected from the group consisting of additional chromatography data and additional optical data.

In another aspect, the present disclosure provides a method of determining a multi-omic profile of a subject, comprising: (a) obtaining chromatography data obtained from a biological sample of the subject; (b) processing, using a machine-learning (ML) algorithm, a set of input features of the chromatography data to generate output data, wherein the set of input features do not comprise a presence or a quantity of a metabolite of the biological sample; and (c) determining the multi-omic profile based at least in part on the output data.

In some embodiments, the method further comprises, (d) processing the metabolic profile to determine a presence or an absence of a disease state. In some embodiments, the processing in (d) further comprises processing a characteristic of the subject. In some embodiments, the characteristic is selected from the group consisting of previous tumor, demographics, clinical characteristic, demographic characteristic, and phenotypic characteristic. In some embodiments, the disease state is selected from the group consisting of an oncological disease, an infectious disease, a chronic disease, a nutritional deficiency, an environmental disease, an autoimmune disorder, and a genetic disease. In some embodiments, the disease state comprises a plurality of disease states, and wherein the method further comprises processing the metabolic profile to determine a presence or absence of each of the plurality of disease states. In some embodiments, the plurality of disease states comprises at least about 5 disease states. In some embodiments, the plurality of disease states comprises at least about 50 disease states. In some embodiments, the presence or the absence of the disease state is determined at an accuracy of at least about 85%. In some embodiments, the presence or the absence of the disease state is determined using a single sample from the subject. In some embodiments, the method further comprises processing the metabolic profile to determine a presence or an absence of use of a compound by the subject. In some embodiments, the biological sample is a urine sample. In some embodiments, the chromatography data is derived from a gas chromatography system. In some embodiments, the chromatography data is derived from a liquid chromatography system. In some embodiments, the set of input features do not comprise a presence or a quantity of an analyte of the biological sample. In some embodiments, the ML algorithm comprises a fuzzy decision network. In some embodiments, the method further comprises repeating (a)-(c) for a plurality of biological samples of a plurality of subjects to generate a plurality of metabolic profiles. In some embodiments, the method further comprises analyzing the plurality of metabolic profiles to determine a differential feature of the plurality of metabolic profiles. In some embodiments, the method further comprises, prior to (a), performing chromatography on the biological sample to generate the chromatography data. In some embodiments, the chromatography does not comprise derivatization of the biological sample. In some embodiments, the biological sample is an unpreserved biological sample. In some embodiments, the unpreserved biological sample is a raw biological sample. In some embodiments, the method further comprises, subsequent to (a), processing the sample with a mass spectrometer. In some embodiments, the method further comprises, determining the presence or the quantity of the metabolite. In some embodiments, the method further comprises determining the metabolic profile based further on the presence or the quantity of the metabolite. In some embodiments, the output data comprises the presence or the quantity of the metabolite. In some embodiments, the chromatography data comprises a first gas chromatography data and a second liquid chromatography data. In some embodiments, the set of input features further comprises additional data. In some embodiments, the additional data comprises additional data selected from the group consisting of additional chromatography data and additional optical data.

In another aspect, the present disclosure provides a system, comprising: a cartridge configured to receive a biological sample from a subject; and a chromatography system configured to receive the cartridge and process at least a portion of the biological sample.

In some embodiments, the cartridge comprises a test strip. In some embodiments, the cartridge comprises a collection cup. In some embodiments, the chromatography system comprises a gas chromatography system. In some embodiments, the chromatography system comprises a liquid chromatography system. In some embodiments, the chromatography system is a single use chromatography system. In some embodiments, the system further comprises a data connection configured to transmit data associated with the at least a portion of the biological sample from the chromatography system. In some embodiments, the data connection comprises a wireless data connection. In some embodiments, the system further comprises an autosampler configured to provide a plurality of cartridges comprising the cartridge to the chromatography system. In some embodiments, the chromatography system comprises one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), and conductivity detector.

In another aspect, the present disclosure provides a system, comprising: a chromatography system configured to generate chromatography data comprising a set of input features from at least a portion of a biological sample of a subject; one or more computer processors operatively coupled to the chromatography system, wherein the one or more computer processors are individually or collectively programmed to process, using a machine-learning (ML) algorithm, the chromatography data to generate output data related to a metabolic profile of the subject.

In some embodiments, the cartridge comprises a test strip. In some embodiments, the cartridge comprises a collection cup. In some embodiments, the chromatography system comprises a gas chromatography system. In some embodiments, the chromatography system comprises a liquid chromatography system. In some embodiments, the chromatography system is a single use chromatography system. In some embodiments, the system further comprises a data connection configured to transmit data associated with the at least a portion of the biological sample from the chromatography system to the one or more computer processors. In some embodiments, the data connection comprises a wireless data connection. In some embodiments, the system further comprises an autosampler configured to provide a plurality of cartridges comprising the cartridge to the chromatography system. In some embodiments, the chromatography system comprises one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), and conductivity detector.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a flowchart of a method of determining a metabolic profile of a subject.

FIG. 2 shows a plot of a similarity assessment generated by a fuzzy decision network on chromatography data.

FIG. 3 shows a plot of a bladder tumor positive vs tumor negative determination performed by an ML algorithm.

FIG. 4 shows a plot of a prostate tumor positive vs tumor negative determination performed by an ML algorithm.

FIG. 5 shows a computer system that is programmed or otherwise configured to implement methods provided herein.

FIG. 6 shows a table of properties of the subjects and samples used in Example 1.

FIGS. 7A-7D show the performance of an ML algorithm at determining a lung cancer state of a plurality of subjects based on a public dataset.

FIG. 8 shows an example of a one-dimensional fuzzy decision tree.

FIG. 9 shows an example of a decision surface with a data overlay plot in two-dimensions.

FIG. 10 shows a schematic of a chromatographic system configured to input data into an ML-algorithm.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

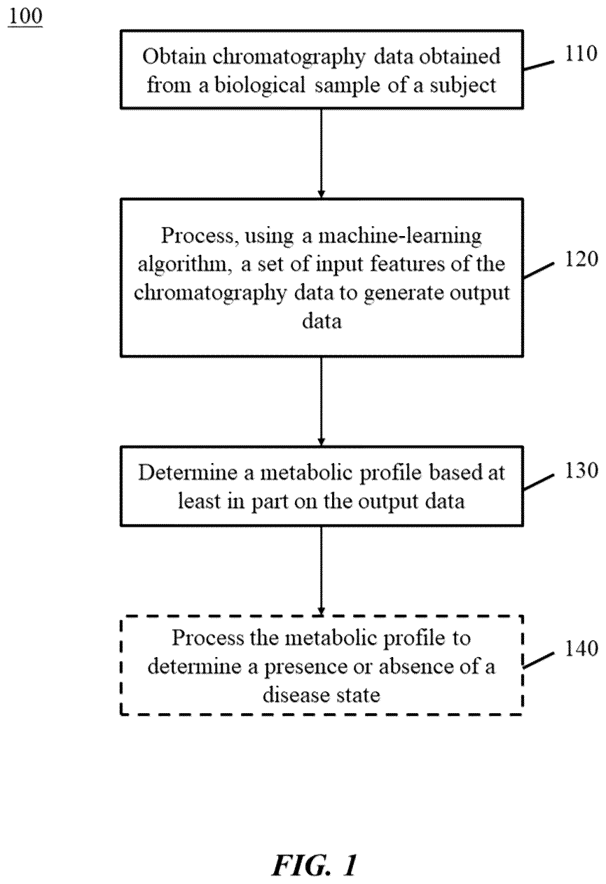

FIG. 1 shows a flowchart of a method 100 of determining a metabolic profile of a subject. In an operation 110, the method 100 can comprise obtaining chromatography data obtained from a biological sample of the subject. Though described herein with respect to metabolic profiling, method 100 may also be used to determine other profiles. For example, a multi-omic profile (e.g., a profile comprising a plurality of data sets (e.g., genomics, proteomics, transcriptomics, metabolomics, epigenomics, microbiomics, or the like, or any combination thereof)) can be generated using the methods and systems of the present disclosure.

The biological sample may be a sample taken from or derived from a sample taken from the subject. The biological sample can be obtained (e.g., extracted or isolated) from or include blood (e.g., whole blood), plasma, serum, urine, saliva, mucosal excretions, sputum, stool, tears, or the like, or any combination thereof. The biological sample can be a fluid or tissue sample (e.g., skin sample). In some examples, the sample is obtained from a cell-free bodily fluid, such as whole blood, saliva, or urine. In some examples, the sample can include circulating tumor cells. The biological sample may be an unpreserved biological sample. For example, the biological sample may not have preservatives added to the biological sample. Use of an unpreserved biological sample may reduce the number of species in the biological sample, which can improve the usability of the data that are produced from the biological sample. For example, the lack of preservatives can reduce the error in the data by reducing the impurity signal generated by the preservatives. Additionally, use of an unpreserved biological sample can simplify the processing of the sample prior to acquisition of the data. For example, not including preservatives in the biological sample can enable an untrained user (e.g., the subject) to acquire the biological sample, which can reduce processing time and other delays. The unpreserved biological sample may be a raw biological sample. A raw biological sample may have no processing (e.g., preserving, concentrating, amplifying, cleaning, etc.) operations performed to the biological sample. The raw biological sample may be a biological sample in a same state as when it was acquired from the subject.

In some cases, the chromatography data can be derived from a gas chromatography system. For example, the chromatography data can be raw data derived from processing a sample in a gas chromatography system. In this example, the chromatography data may be raw chromatography data. The chromatography data may be derived from a liquid chromatography system. For example, the sample can be processed by a liquid chromatography system, and the readout from that processing can be the chromatography data. The liquid chromatography system may be a high-performance liquid chromatography system.

In some cases, prior to operation 110, the chromatography can be performed on the biological sample to generate the chromatography data. For example, the biological sample can be collected and inserted without processing into a chromatography instrument to generate the chromatography data. In some cases, the chromatography may not comprise derivatization of the biological sample. In this example, the biological sample may be unprocessed (e.g., in a raw state) when the chromatography is performed.

The chromatography data can comprise a plurality of chromatography data. The plurality of chromatography data can comprise data from a same type of chromatography. For example, the plurality of chromatography data can comprise a plurality of gas chromatograms. The plurality of chromatography data can comprise a plurality of chromatography data from different types of chromatography. For example, the plurality of chromatography data can comprise chromatography data from a gas chromatograph and a liquid chromatograph. The plurality of chromatography data may be from a same sample. For example, a single urine sample can be processed by both gas and liquid chromatography. The plurality of chromatography data may be from different samples from the subject. For example, a urine sample and a blood sample can be processed to generate chromatography data. In some cases, additional data can be provided with the chromatography data. Examples of additional data include, but are not limited to, data generated by nuclear magnetic resonance (NMR), optical spectroscopy (e.g., absorption, fluorescence, Raman spectroscopy, etc.), mass spectroscopy, electronic techniques (e.g., cyclic voltammetry, impedance measurements, etc.), or the like, or any combination thereof.

In some cases, subsequent to operation 110, the method may comprise processing the sample with a mass spectrometer. For example, the chromatography data can be acquired, and the sample can be input into a mass spectrometer as the components of the sample exit the chromatography machine. In some cases, the data from the mass spectrometer can be input into the machine-learning algorithm of operation 120. In some cases, the data from the mass spectrometer may not be input into the machine-learning algorithm of operation 120.

In an operation 120, the method 100 can comprise processing, using a machine-learning (ML) algorithm, a set of input features of the chromatography data to generate output data. The set of input features may not comprise a presence or a quantity of a metabolite of the biological sample. For example, the set of input features may comprise raw data from the sample, but not a tag noting the presence of a metabolite.

The set of input features may not comprise a presence or a quantity of an analyte of the biological sample. The analyte may be a non-metabolically active species. For example, the analyte may be a molecule that passed through the body of the subject without undergoing a metabolic process. In this example, the analyte can be indicative of the metabolic processes occurring in the subject. For example, a presence of a non-metabolized drug in a subject's urine can indicate that the metabolic pathways that process the drug are not occurring in the subject. The output data may comprise the presence or the quantity of the metabolite. For example, the ML algorithm can identify a metabolite related to a cancer state of the subject, and the presence of that metabolite can be used later in the method to help determine the cancer state of the subject. The ML algorithm can determine the presence or quantity of the metabolite from input data that does not directly comprise the presence or quantity of the metabolite. For example, the ML algorithm can determine that a peak in a chromatogram corresponds to a given metabolite, despite the chromatographic data not directly indicating the presence of the metabolite.

In some cases, the ML algorithm comprises a fuzzy decision network. A fuzzy decision network can comprise a tree comprising one or more nodes. Nodes higher (e.g., earlier) in the tree can be weighted to represent higher degrees of variance, while lower nodes in the tree can be weighted for lower variance, and terminal nodes can represent complete solutions to the problem provided to the fuzzy decision network (e.g., a classification problem). The use of the fuzzy decision network can provide for efficient processing of data. Each terminal node of the network can contain a set of earlier nodes that may comprise the attributes and constraints that define an interaction pattern common to the data mapped to the terminal node. For example, all of the data mapped to a particular terminal node can have similar attributes and constraints that resulted in that data being mapped to that node. The fuzzy decision network can be at least a portion of a multi-dimensional fuzzy decision network. For example, the fuzzy decision network can operate on a plurality of dimensions of the input data. The use of the fuzzy decision network can provide efficient processing of convoluted data (e.g., data where the elements of the data have correlations).

Machine learning algorithms implemented on a local computer or a remote server can process the set of input features. For example, a machine learning algorithm can be configured to process input data to generate output data (e.g., categorized data, tagged data, etc.). A different machine learning can be trained to identify metabolic profiles based at least in part on the output data. Other machine learning algorithms can be configured to identify a disease or disorder state based at least in part on the metabolic profile. Still other machine learning algorithms can be configured to identify a metabolic state of the subject.

The machine learning algorithms can be supervised, semi-supervised, or unsupervised. A supervised machine learning algorithm can be trained using labeled training inputs, e.g., training inputs with known outputs. The training inputs can be provided to an untrained or partially trained version of the machine learning algorithm to generate a predicted output. The predicted output can be compared to the known output, and if there is a difference, the parameters of the machine learning algorithm can be updated. A semi-supervised machine learning algorithm can be trained using a large number of unlabeled training inputs and a small number of labeled training inputs. An unsupervised machine learning algorithm, e.g., a clustering algorithm, can find previously unknown patterns in data sets without pre-existing labels.

One example of a machine learning algorithm that can perform some of the functions described above, e.g., generating output data, is a neural network. Neural networks can employ multiple layers of operations to predict one or more outputs, e.g., metabolic data, from one or more inputs, e.g., chromatography data sets. Neural networks can include one or more hidden layers situated between an input layer and an output layer. The output of each layer can be used as input to another layer, e.g., the next hidden layer or the output layer. Each layer of a neural network can specify one or more transformation operations to be performed on input to the layer. Such transformation operations may be referred to as neurons. The output of a particular neuron can be a weighted sum of the inputs to the neuron, adjusted with a bias and multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the algorithm's weights and biases to account for the difference between the predicted outputs and the expected outputs. Specifically, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training can be complete when the predicted outputs satisfy a convergence condition, such as obtaining a small magnitude of calculated cost.

Convolutional neural networks (CNNs) and recurrent neural networks can be used to classify or make predictions from output data. CNNs are neural networks in which neurons in some layers, called convolutional layers, receive data from only small portions of an chromatogram. These small portions may be referred to as the neurons' receptive fields. Each neuron in such a convolutional layer can have the same weights. In this way, the convolutional layer can detect features, e.g., metabolic states, in any portion of the input chromatogram.

RNNs, meanwhile, are neural networks with cyclical connections that can encode dependencies in time-series data, e.g., chromatography data taken at various time points. An RNN can include an input layer that is configured to receive a sequence of time-series inputs. An RNN can also include one or more hidden recurrent layers that maintain a state. At each time step, each hidden recurrent layer can compute an output and a next state for the layer. The next state can depend on the previous state and the current input. The state can be maintained across time and can capture dependencies in the input sequence. Such an RNN can be used to track progress of a particular metabolic or disease state in a subject.

One example of an RNN is a long short-term memory network (LSTM), which can be made of LSTM units. An LSTM unit can be made of a cell, an input gate, an output gate, and a forget gate. The cell can be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit. The activation function of the LSTM gate can be the logistic function.

Other examples of machine learning algorithms that can be used with the present disclosure are regression algorithms, decision trees, support vector machines, Bayesian networks, clustering algorithms, reinforcement learning algorithms, and the like.

In an operation 130, the method 100 can comprise determining the metabolic profile based at least in part on the output data. The metabolic profile may comprise an indication of a presence or absence of one or more analytes, an indication of a metabolic state of the subject, data related to the metabolic state of the subject, or the like, or any combination thereof.

The method may comprise processing the metabolic profile to determine a presence or an absence of a use of a compound by the subject. For example, the metabolic profile can be processed to determine if the subject is taking steroids. In another example, the metabolic profile can be processed to determine if the subject is using illicit drugs. In another example, the metabolic profile can be processed to determine an efficacy of a pharmaceutical intervention the subject is using.

In some cases, operations 110-130 may be repeated for a plurality of biological samples of a plurality of subjects to generate a plurality of metabolic profiles. For example, a plurality of subject can provide a plurality of urine samples, and each of the urine samples can be processed according to operations 110-130. The plurality of metabolic profiles can be analyzed to determine a differential feature of the plurality of metabolic profiles. For example, the plurality of urine samples can be analyzed to determine a differential feature indicative of a cancer state in a subset of the subjects of the plurality of subjects. Examples of differential features include, but are not limited to, a presence or absence of an analyte, an amount of an analyte present, proportions of different analytes to one another, or the like, or any combination thereof.

In some cases, the presence or quantity of the metabolite can be determined. For example, the metabolic profile may comprise information related to the presence or quantity of the metabolite. In this example, the metabolic profile can indicate the presence or quantity of the metabolite, thereby providing additional information to a user interpreting the metabolic profile. In some cases, the presence or quantity of the metabolite can be determined at least in part based on the output data. The metabolic profile can be determined based further on the presence or quantity of the metabolite. For example, the metabolic profile can be refined using the information related to the presence or quantity of the metabolite.

In some cases, in an optional operation 140, the method 100 can comprise processing the metabolic profile to determine a presence or an absence of a disease state. In some cases, the processing in optional operation 140 comprises processing a characteristic of the subject. Examples of characteristics include, but are not limited to, medical history (e.g., history of a presence of a cancer or tumor, history of treatments, etc.), demographics (e.g., information about the subject, information about the residence location of the subject, etc.), clinical characteristics, phenotypic characteristics (e.g., characteristics derived from the subject's phenotype), or the like, or any combination thereof.

The disease state may be related to an abnormal condition, or a disorder of a biological function or a biological structure (e.g., an organ), that can affect part or all of a subject. A disease state may be caused by factors originally from an external source (e.g., an infectious disease). An infectious disease may result from a presence or previous presence of one or more pathogenic microbial agents (e.g., viruses, bacteria, fungi, protozoa, multicellular organisms, prions, etc.) A disease state may be caused by internal disfunctions of the subject (e.g., an autoimmune disease, cancer, genetic diseases, etc.). Examples of cancers include, but are not limited to, lung cancer, thyroid cancer, colorectal cancer, breast cancer, prostate cancer, rectal cancer, chronic lymphatic leukemia, pancreatic cancer, and ovarian cancer. A disease state may correspond to an oncological disease (e.g., a cancer), an infectious disease (e.g., a disease state caused at least in part by a pathogen), a chronic disease (e.g., a disease that is present over a long period of time), a nutritional deficiency (e.g., a lack of a nutrient intake by the subject), an environmental disease (e.g., exposure to pollutants), an autoimmune disorder (e.g., allergies, acquired immunodeficiency syndrome, etc.), a genetic disease (e.g., a disease state caused at least in part by a portion of the subject's genome), or the like.

The metabolic profile may be processed to determine a presence or an absence of each of a plurality of disease states comprising the disease state. In some cases, the disease state can comprise a plurality of disease states. The method can comprise processing the metabolic profile to determine a presence or absence of each of the plurality of disease states. For example, a single set of chromatography data can be processed to determine a presence or absence of a plurality of disease states. In this example, the determination of the different disease states can be related to different portions of the chromatography data and/or different ML algorithms used. The metabolic profile may be processed to determine a presence or absence of at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more disease states. The metabolic profile may be processed to determine a presence or absence of at most about 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, or fewer disease states. The metabolic profile may be processed to determine a presence or absence of a number of disease states in a range as defined by any two of the preceding values. For example, the metabolic profile can be processed to determine a presence or absence of about 10 to about 50 disease states.

The presence or absence of the disease state can be determined at an accuracy, sensitivity, and/or specificity of at least about at least about 10%, 20%, 30%, 40%, 50%, 60%, 70%, 80%, 85%, 90%, 91%, 92%, 93%, 94%, 95%, 96%, 97%, 98%, 99%, 99.5%, 99.9% or more. The presence or absence of the disease state can be determined at an accuracy, sensitivity, and/or specificity of at least about at most about 99.9%, 99.5%, 99%, 98%, 97%, 96%, 95%, 94%, 93%, 92%, 91%, 90%, 85%, 80%, 70%, 60%, 50%, 40%, 30%, 20%, 10%, or less. The presence or absence of the disease state can be determined at an accuracy, sensitivity, and/or specificity in a range as defined by any two of the preceding values.

The presence or absence of the disease state may be determined using a single sample from the subject. For example, a single urine sample can be used to determine the presence or absence of a cancer in the subject. In some cases, the presence or absence of the disease state can be determined using a plurality of samples from the subject. The plurality of samples may be at most about 10, 9, 8, 7, 6, 5, 4, 3, or fewer samples.

In another embodiment, the present disclosure provides a system. The system can comprise a cartridge configured to receive a biological sample from a subject. The system can comprise a chromatography system configured to receive the cartridge and processes at least a portion of the biological sample. The system may be configured to at least partially implement the methods of the present disclosure.

The cartridge may comprise a test strip, collection cup, port configured to receive the biological sample from another collection device, or the like, or any combination thereof. For example, the cartridge may comprise a test strip configured to absorb urine into the cartridge. In another example, the cartridge can be configured to receive a needle to transfer blood from a syringe to the test cartridge. In some cases, the chromatography system comprises a gas chromatography system (e.g., a system configured to analyze the sample using gas chromatography). In some cases, the chromatography system comprises a liquid chromatography system (e.g., a system configured to analyze the sample using liquid chromatography). In some cases, the liquid chromatography system is a high performance liquid chromatography system.

The chromatography system may be a single use chromatography system. For example, the chromatography system may be configured to process a single biological sample prior to being discarded. The chromatography system may be a multi-use chromatography system. For example, the chromatography system may be configured to process a plurality of biological samples prior to being discarded or repaired.

The system may comprise a data connection configured to transmit data associated with the at least a portion of the biological sample from the chromatography system. For example, the data connection can be configured to transmit chromatography data from the system to one or more computer processors configured to process the chromatography data using an ML algorithm. In another example, the data connection can be configured to transmit a metabolic profile to a user for interpretation. In some cases, the data connection comprises a wireless connection (e.g., a cellular wireless data connection, a Bluetooth® connection, a Wi-Fi connection, an ultrawideband connection, etc.).

The system may comprise an autosampler configured to provide a plurality of cartridges comprising the cartridge to the chromatography system. The autosampler can be configured with a receiving portion configured to receive one or more cartridges, a storage portion configured to hold one or more cartridges, or the like, or any combination thereof.

The chromatography system may comprise one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), conductivity detector, or the like, or any combination thereof.

In another aspect, the present disclosure may provide a system comprising a chromatography system configured to generate chromatography data comprising a set of input features from at least a portion of a biological sample of a subject, as described elsewhere herein. The system may comprise one or more computer processors operatively coupled to the chromatography system. The one or more computer processors can be individually or collectively programmed to processes, using a ML algorithm, the chromatography data to generate output data related to a metabolic profile of the subject. The system may be configured to at least partially implement the methods of the present disclosure.

The cartridge may comprise a test strip, collection cup, port configured to receive the biological sample from another collection device, or the like, or any combination thereof. For example, the cartridge may comprise a test strip configured to absorb urine into the cartridge. In another example, the cartridge can be configured to receive a needle to transfer blood from a syringe to the test cartridge. In some cases, the chromatography system comprises a gas chromatography system (e.g., a system configured to analyze the sample using gas chromatography). In some cases, the chromatography system comprises a liquid chromatography system (e.g., a system configured to analyze the sample using liquid chromatography). In some cases, the liquid chromatography system is a high performance liquid chromatography system.

The chromatography system may be a single use chromatography system. For example, the chromatography system may be configured to process a single biological sample prior to being discarded. The chromatography system may be a multi-use chromatography system. For example, the chromatography system may be configured to process a plurality of biological samples prior to being discarded or repaired.

The system may comprise a data connection configured to transmit data associated with the at least a portion of the biological sample from the chromatography system. For example, the data connection can be configured to transmit chromatography data from the system to one or more computer processors configured to process the chromatography data using an ML algorithm. In another example, the data connection can be configured to transmit a metabolic profile to a user for interpretation. In some cases, the data connection comprises a wireless connection (e.g., a cellular wireless data connection, a Bluetooth® connection, a Wi-Fi connection, an ultrawideband connection, etc.).

The system may comprise an autosampler configured to provide a plurality of cartridges comprising the cartridge to the chromatography system. The autosampler can be configured with a receiving portion configured to receive one or more cartridges, a storage portion configured to hold one or more cartridges, or the like, or any combination thereof.

The chromatography system may comprise one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), conductivity detector, or the like, or any combination thereof.

### Computer Systems

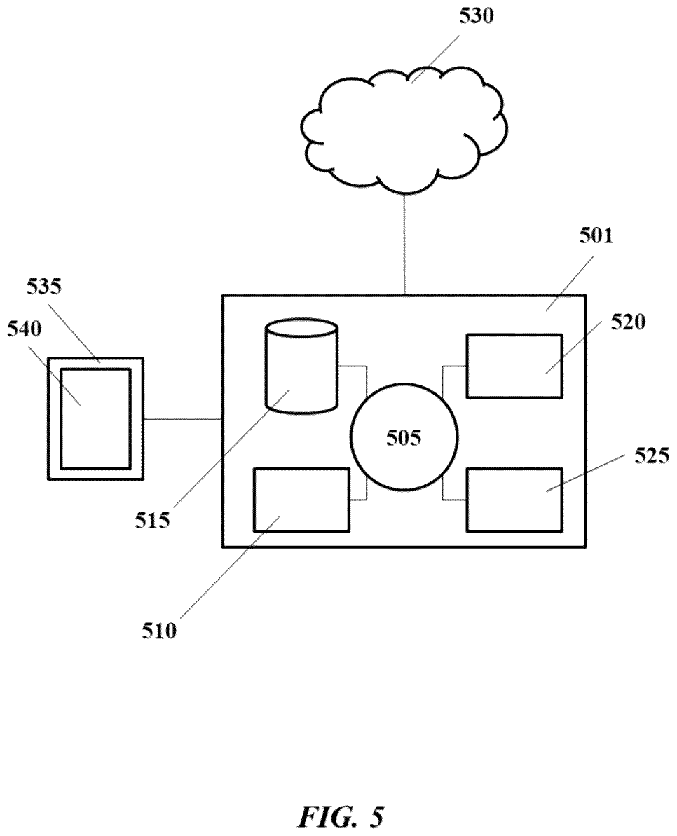

The present disclosure provides computer systems that are programmed to implement methods of the disclosure. FIG. 5 shows a computer system 501 that is programmed or otherwise configured to implement the methods of the present disclosure. The computer system 501 can regulate various aspects of the present disclosure, such as, for example, acquisition of chromatographic data, analysis via ML algorithm, or the like, or any combination thereof. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user. Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, an output based at least in part on an ML processed chromatogram. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, be a machine learning algorithm as described elsewhere herein.

The following examples are illustrative of certain systems and methods described herein and are not intended to be limiting.

### EXAMPLES

### Example 1

### Detection of Bladder, Prostate, and Lung Cancer Via Urine Samples

Using methods and systems of the present disclosure, bladder and prostate cancer were detectable via metabolic profiling of urine samples. Urine samples from 20 symptomatic and 15 healthy individuals were obtained. The samples were derivatized by a) incubating with urease for 30 minutes to reduce the amount of urea in the sample, b) extracted with ethanol and/or methanol, centrifuged, and the resultant pellet was discarded, and c) methoxamine hydrochloride was added to the supernatant and incubated for up to two hours, then incubated with N-methyl-N-(trimethylsilyl)trifluoroacetamide and 1% Trimethylsilyl chloride.

To generate chromatography data, helium carrier gas was injected at 1.5 milliliters/minute with a split ratio of 1:20 into an Agilent gas chromatography/Pegasus mass spectroscopy instrument. The injector, transfer line, and ion source were maintained at temperatures of 220° C., 200° C., and 250° C., respectively. The oven was programed to start at 70° C. for 0.2 minutes, then ramp to 270° C. (10 minute hold at temperature) at a rate of 10° C./minute. The mass spectrometry ionization was set to electron ionization (70 eV), and a mass spectrogram was taken for a mass/charge ratio of 40 to 600 with a solvent cutoff of 360 seconds and an acquisition rate of 20hertz.

A subset of the resultant chromatography data was processed into a ML disease model for bladder cancer. Different portions of the chromatography data were processed to generate the ML model, test and evaluate the standard deviation of the model, and used as blinded samples to validate the model and determine the sensitivity and specificity of the model.

For the 35 test samples, a leave-one-out cross validation processes was employed where a single sample was left out of the model creation and then characterized using the model generated from the other 34 test samples. Since the model was generated without any data from the left out sample, that sample represents a true unknown with respect to the profile that is generated.

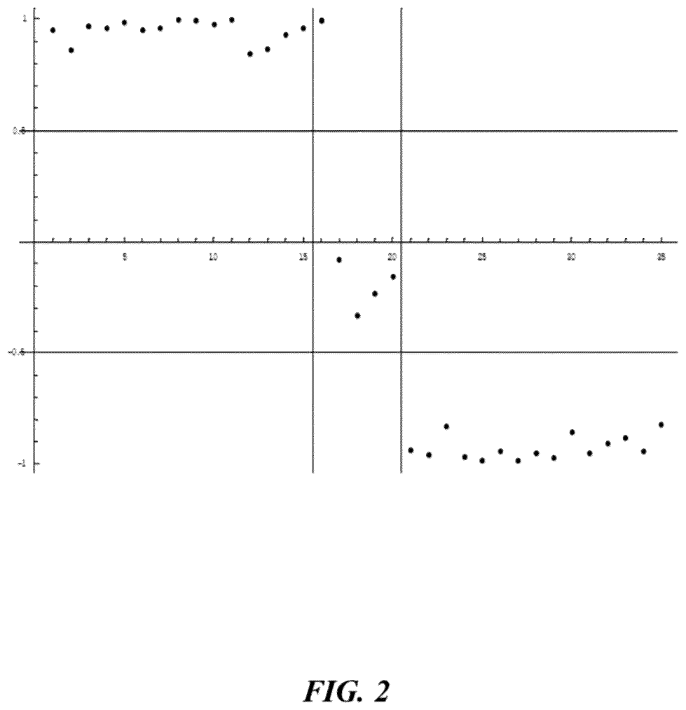

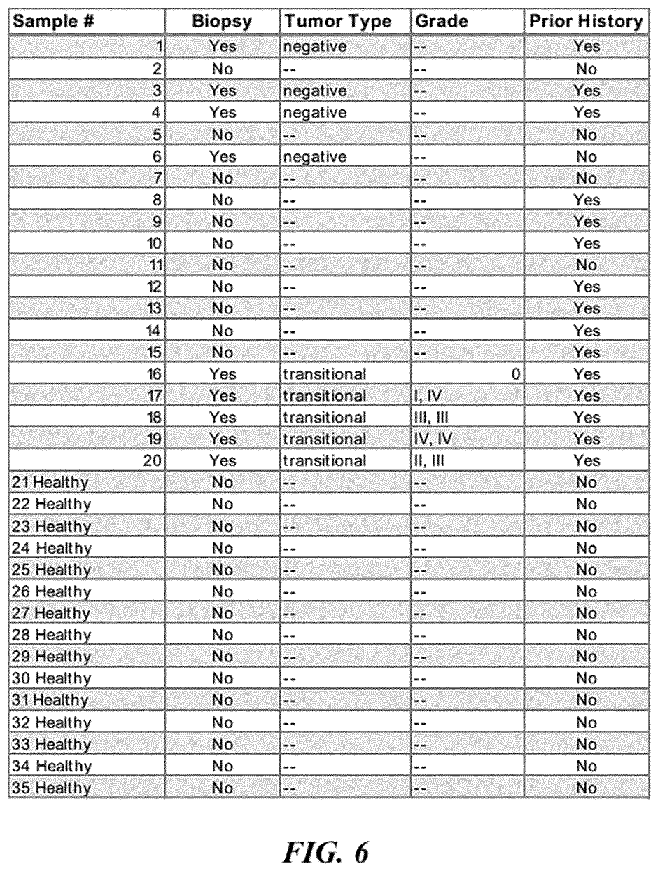

FIG. 6 shows a table of properties of the subjects and samples used in Example 1. FIG. 2 shows a plot of a similarity assessment generated by a fuzzy decision network on chromatography data. The first twenty samples (numbered 1-20) are from the subjects who were symptomatic for bladder cancer, while the remaining samples (numbered 21-35) were asymptomatic subjects. The symptomatic patients underwent cystoscopies with the exception of sample 15. Biopsies were taken from patients 1, 3, 4, 6, and 16-20. The symptomatic samples except 2, 5, 6, 7, and 11 were from patients with a prior history of bladder cancer. As can be seen from FIG. 2, the identification provided unambiguous separation (e.g., no false positives or negatives) for both the symptomatic subjects and the asymptomatic subjects.

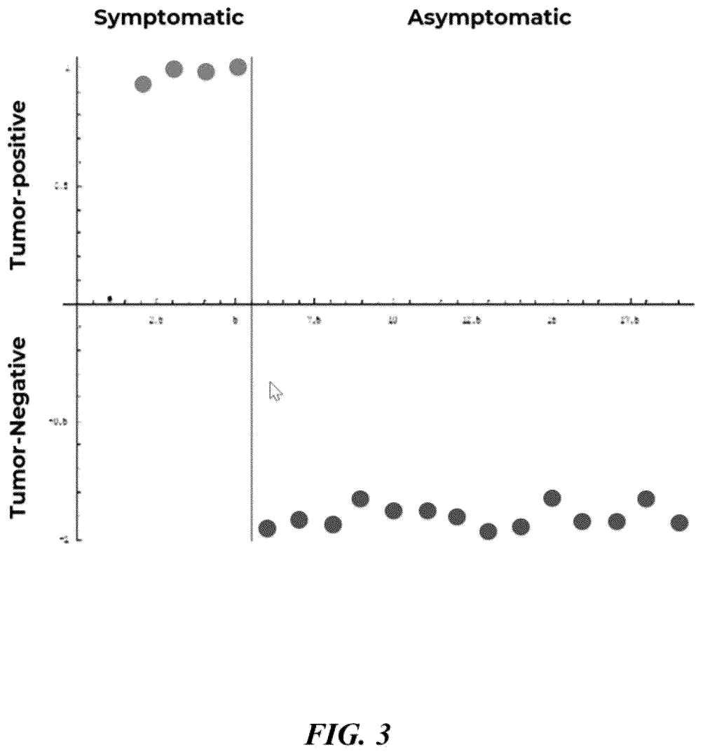

FIG. 3 shows a plot of a bladder tumor positive vs tumor negative determination performed by an ML algorithm. Using the same data as FIG. 2, an additional ML algorithm was trained to discriminate tumor positive from tumor negative subjects. Using the same leave-one-out style cross validation, the ML algorithm was able to separate the tumor positive from tumor negative subjects with unity specificity and sensitivity.

The ML algorithms can provide early detection of a cancer state (e.g., the tumor of sample 16 in FIG. 2 was graded stage 0). The ML algorithms can additionally provide differentiation between different stages of the disease the algorithm is trained for (e.g., the different similarities for the symptomatic samples in FIG. 2). Non-invasive, accurate and early detection of a cancer state can significantly improve outcomes for patients with a disease, and the chromatography trained ML algorithms of the present disclosure can provide these benefits.

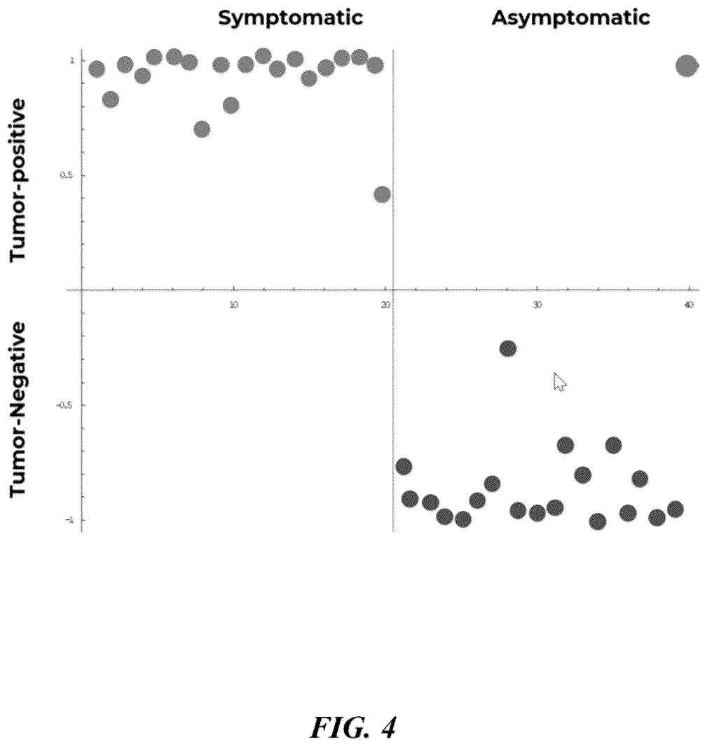

FIG. 4 shows a plot of a prostate tumor positive vs tumor negative determination performed by an ML algorithm. Using chromatography data generated in the same way as for FIGS. 2-3, a cohort of 40 patients (20 known tumor positive samples and 20 expected healthy controls) was investigated by ML analysis. The ML algorithm was able to identify a cancer positive sample from one of the expected healthy controls who presented a prostate specific antigen test of <1 nanogram/milliliter. Thus, the ML algorithm demonstrates an ability to discern a cancer state that may be missed by other screening techniques.

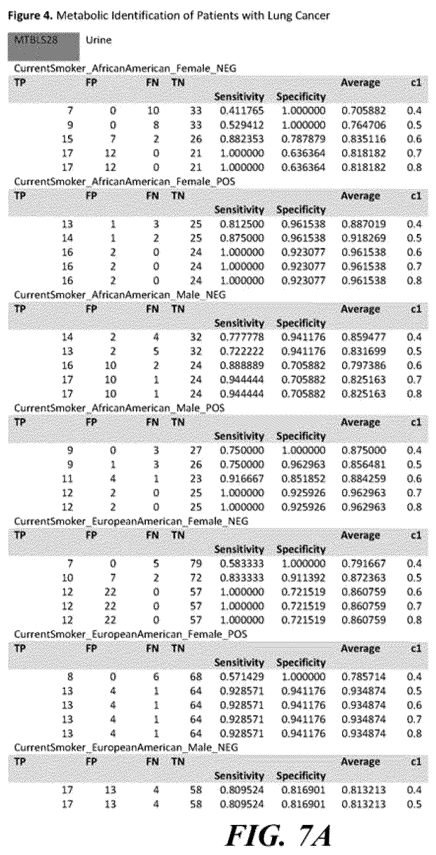

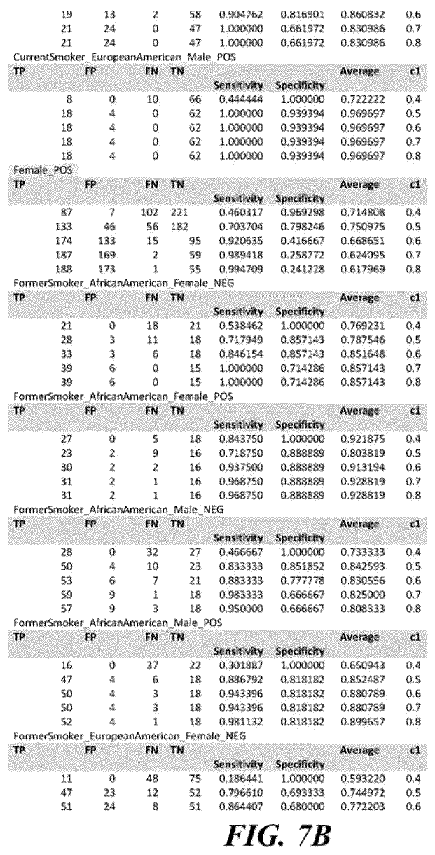

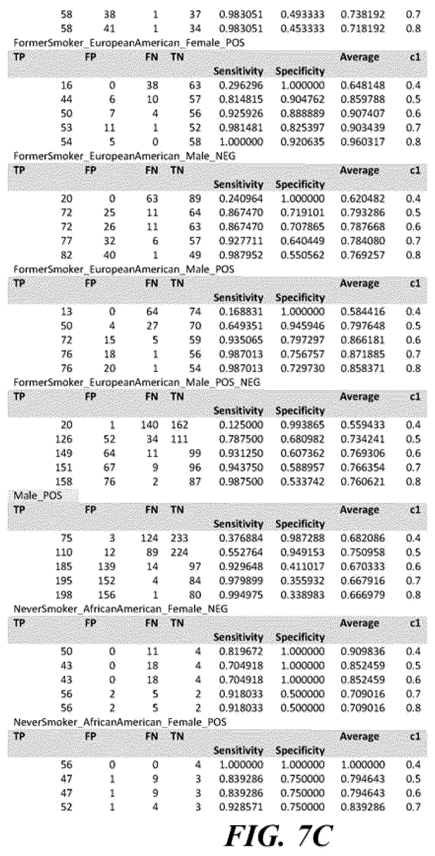

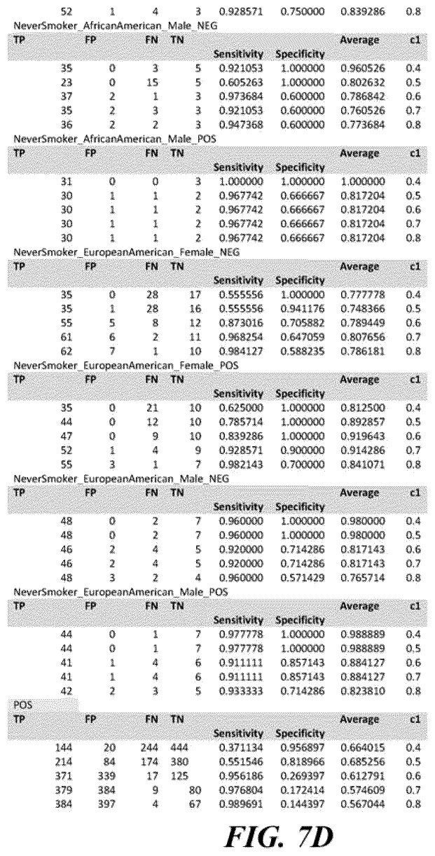

FIGS. 7A-7D show the performance of an ML algorithm at determining a lung cancer state of a plurality of subjects based on a public dataset. The data used to generate the ML algorithms were taken from a publicly available lung cancer dataset, and a plurality of different ML algorithms were generated based on the data set. The TP, FP, FN, and TN columns of FIGS. 7A-7D represent the number of true positive, false positive, false negative, and true negative results for a given ML algorithm, respectively. These values are used to calculate the sensitivity and specificity of the ML algorithm, which can be averaged to provide a metric for evaluating the ML algorithm at different confidence intervals, c1.

Additional information related to this example can be found in A. Jemal, R. Siegel, J. Xu, and E. Ward, “Cancer statistics, 2010, ” CA Cancer Journal for Clinicians, vol. 60, no. 5, pp. 277-300, 2010; D. J. McConkey, S. Lee, W. Choi et al., “Molecular genetics of bladder cancer: emerging mechanisms of tumor initiation and progression,” Urologic Oncology, vol. 28, no. 4, pp. 429-440, 2010; M. S. Cookson, H. W. Herr, Z. F. Zhang, S. Soloway, P. C. Sogani, and W. R. Fair, “The treated natural history of high risk superficial bladder cancer: 15-year outcome,” Journal of Urology, vol. 158, no. 1, pp. 62-67, 1997; M. S. Soloway, M. Sofer, and A. Vaidya, “Contemporary management of stage Tl transitional cell carcinoma of the bladder,” Journal of Urology, vol. 167, no. 4, pp. 1573-1583, 2002; Mufti G R, Singh M. “Value of random mucosal biopsies in the management of superficial bladder cancer,” Eur. Urol. 22:289-93, 1992 Kriegmair M., Baumgartner R, Lumper W, Waidelich R, Hofstetter A. “Early clinical experience with 5-aminolevulinic acid for the photodynamic therapy of superficial bladder cancer,” Br. J. Urol 155:105-9, 1996 G. Bepler, M. Begum, and G. R. Simon, “Molecular analysis-based treatment strategies for non-small cell lung cancer,” Cancer Control, vol. 15, no. 2, pp. 130-139, 2008; David J Sullivan Jr, Nikola Kaludov and Martin N. Martinov, “Discovery of Potent, Novel, Non-toxic Anti-malarial Compounds via Quantum Modeling, Virtual Screening and In Vitro Experimental Validation,” Malaria Journal 2011, 10:274 Breiman L. “Heuristics of instability and stabilization in model selection,” Ann Stat 1996; 24:2350-83; Braga-Neto U M, Dougherty ER. “Is cross-validation valid for small-sample microarray classification?” Bioinformatics 2004 ; 20:374-80; Shariat S F, Karakiewicz P I, Ashfaq R, Lerner S P, Palapattu G S, Cote R J, Sagalowsky A I, Lotan Y. “Multiple biomarkers improve prediction of bladder cancer recurrence and mortality in patients undergoing cystectomy,” Cancer. 2008 Jan. 15; 112 (2):315-25; Bolenz C, Lotan Y. “Molecular biomarkers for urothelial carcinoma of the bladder: challenges in clinical use,” Nat Clin Pract Urol. 2008 December; 5 (12):676-85; Anderson NL, Anderson NG. “The human plasma proteome—history, character, and diagnostic prospects,” Mol Cell Proteomics 2002; 1:845-67; Pearson H. “Meet the human metabolome,” Nature 2007; 446:8; Bujak R, Daghir E, Rybka J, Koslinski P, Markuszewski MJ. “Metabolomics in urogenital cancer,” Bioanalysis. 2011 April; 3 (8): 913-23; each of which is incorporated by reference in its entirety.

### Example 2

### Determination of a Disease State in a Subject by Use of a Digital Metabolomic Representation

When an organism (e.g., person, animal, etc.) becomes ill (e.g., has a disease state) their body can change based on the nature of the disease state, which can be reflected in the operation of the organism's metabolic system. The metabolic system can be expressed in the analytes present in excretions from the organism (e.g., urine, feces, etc.), which can be analyzed using, for example, data gathering, disease state model creation, digital sample processing, or the like, or any combination thereof to determine the disease state of the organism. Use of chromatography (e.g., gas chromatography, liquid chromatography, etc.) on samples taken from the organism (e.g., urine, blood, etc.) can enable created of a digital representation of the organism's metabolic profile. The chromatogram generated by the chromatography can comprise a record (e.g., a graph, a set of data points, etc.) showing the results of separating the components of a mixture (e.g., sample). An ML model can be utilized to generate a computational model of a disease state based at least in part on chromatograms of samples from patients diagnosed with the disease state, and the computational models can then be used to aid in the diagnosis of, provide indications of, or diagnose the presence of the disease in unknown samples. The methods and systems of the present disclosure can enable use of a single chromatogram derived from a single sample to find indications of a plurality of diseases from the single sample.

In some examples, the process of generating a disease state model can begin with collection of one or more samples collected from a patient diagnosed with, but not yet treated for the disease state. Chromatography data of the sample can be generated using a chromatography instrument and used as a training data set to develop and train a ML model for the disease state. The ML model can be validated and tested against the data by, for example, a leave-one-out approach. Once the model is generated, samples can be blindly tested to determine a sensitivity, specificity, accuracy, or the like, of the model for the disease state. For example, samples from patients with an unknown presence of absence of the disease state can be analyzed and the results collected and tabulated for statistical analysis of, for example, false positive and false negative rates. Once the ML model is determined to be of sufficient accuracy, sensitivity, or specificity for use, it can be used to screen additional subjects for a presence or absence of the disease state.

The use of chromatography data for disease state identification can provide benefits over genetic sequencing-based technologies. For example, chromatography can provide much faster, more accessible, and lower cost data generation than genetic sequencing, which can, in turn, provide faster turnaround of disease state determination, improved accessibility, and the ability to provide point of care testing. Use of urine samples can provide simpler sample collection than blood, biopsy, or feces samples, allowing an untrained user to collect samples. The ML based disease state determination of the present disclosure can provide an indication of a likelihood of a given disease state, rather than an indication of a class of diseases, improving specificity of the indication and providing granular, actionable information related to particular disease states. For example, a ML based determination can provide an indication of a presence of bladder cancer, lung cancer, or pancreatic cancer rather than an indication that cancer is present. In another example, a presence of an autoimmune disease, infectious disease, and nutrient deficiency can all be determined from a single sample. The ML based determination of disease state may not comprise determination of the precise analytes present in the sample, but rather use the pattern of the entire metabolic profile of the subject. For example, the ML based determination of prostate cancer may not specifically identify a presence of the prostate specific antigen (PSA), but can use the entire metabolic state of the subject, including a presence of PSA, to determine the presence of prostate cancer. The determination of the disease state can also occur earlier than other detection methodologies due to the sensitivity of the body's metabolic processes to a disease state. For example, the metabolic state of a subject can begin to change prior to time when a tumor has grown to a size detectable by ultrasonography.

Testing for the disease state can be performed periodically to determine an efficacy of a treatment of the disease state, an impact of a treatment on the subject (e.g., is the treatment causing side effects, etc.), or the like, or any combination thereof. This can provide real time management of a treatment regimen, which can improve outcomes by both testing treatment efficacy and determining a presence of a negative side effect earlier on. The ML based methods of the present disclosure can also be used in athletic performance monitoring and/or management. For example, a baseline metabolic profile can be established based on samples provided by athletes in a given field and the baseline metabolic profile can be used to test athletes for, for example, performance enhancing drugs. The baseline metabolic profile may also be used to determine an athlete's preparedness level, monitor athletic conditioning and/or health, compare historical metabolic profiles to actual performance, analyze metabolic profile changes based on different training regimens, or the like, or any combination thereof. Similarly, a metabolic profile can be used to enable health and wellness monitoring and management. For example, using a baseline metabolic profile generated from healthy subjects, an individual's metabolic profile can be compared to a health target, enabling monitoring of changes in the individual's metabolic profile based on, for example, nutrition, exercise, medication, etc. Additionally, a subject's metabolic profile can be monitored in times of feeling well and feeling ill, and the comparison of the metabolic profile in those times can provide information related to, for example, the reason for the subject feeling unwell.

### Example 3

### Fuzzy Decision Networks

The fuzzy decision network can be optimized for investigating biomolecular interactions and pathways, and can use metric based machine learning techniques for modeling biomolecular interactions and/or pathways. Systems biology can be a field related to the empirical research of complex interactions in biological systems using a holistic approach. Phenotypic studies can reveal biological network structures that can be modeled and interpreted using mathematical graph theory. Graph-theoretical problems can be complex and present unique algorithmic challenges that may be impractical to achieve with exhaustive computation solutions, especially in personalized data interpretation.

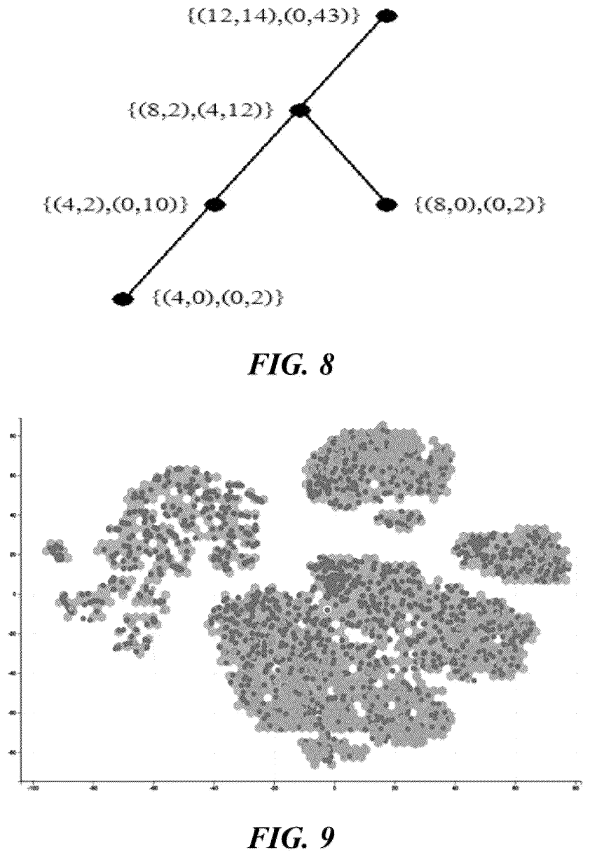

Metabolomics can provide an unbiased, data driven, and hypothesis generating technique that can holistically explore a biological system. Utilization of a suitable modeling architecture with efficient information extraction can provide an ability to develop personalized and predictive profiles. A fuzzy decision network can provide such an architecture. Utilization of a localized metric in the modeling space can allow for rigorous mathematical techniques to be used and can provide a mechanistic hypothesis for the modeled interactions. As the number and complexity of modeled interactions increases (e.g., with inclusion of multiple biochemical constraints), the descriptions of the interactions can be made with a corresponding number of mechanistic attributes. In an example, the modeling process of a fuzzy decision network can produce a fuzzy decision tree (e.g., FIG. 8 shows an example of a one-dimensional fuzzy decision tree). Each node of the fuzzy decision tree can correspond to a single attribute (e.g., interaction constraint), and the weighted attribute (e.g., weighted to represent the highest degree of data variance) can occupy the highest node. A fully resolved fuzzy decision tree can contain terminal nodes presenting a complete solution to the classification problem. Each terminal node can be fully characterized by associated confidence intervals, among other parameters. A model in the form of a fuzzy decision tree can provide an ability to efficiently interpret a data set. Each tree path that contains an active terminal node can also contain a set of nodes with the corresponding attributes and constraints that define the interaction pattern common to all objects mapped to that terminal.

A multi-dimensional generalization of the fuzzy decision tree can be a fuzzy decision network. The increased complexity of the fuzzy decision network can provide a robust underlying logical architecture that can be used to detect and interpret highly convoluted and intricate interaction patterns. A decision network can provide a complete characterization of the interaction patterns found within the modeling data. FIG. 9 shows an example of a decision surface with a data overlay plot in two-dimensions. The generative process of model creation may involve construction of a weighted mathematical graph, and may not be automated. Once a predictive fuzzy decision network is obtained, it may be easily fine-tuned by augmentation with additional data. This may provide an iterative data driven process to generate a natural and unbiased way of improving the predictive resolution of the model by introducing new modeling attributes and individual characteristics personalized to the subject. A plurality of modeling datasets for multiple disease states can be used to build a portfolio of corresponding predictive models.

Additional information related to this example can be found in “Discovery of New Chemical Entities for Alzheimer's Disease Tauopathy,” IR43AG053137-01A1 NIH/NIA phase I SBIR, Sep. 1, 2016-08/31/2018; “A Quantum Similarity Approach for Discovery of Anti-Trypanosome Lead Drugs,” 1R43AI114078-01 NIH/NIAID phase I SBIR, May 15, 2014-04/30/2016; “Discovery of Novel Anti-psychotics through Quantum Similarity,” 1R43MH101892-01, NIH/NIMH, Sep. 1, 2013-Jul. 31, 2015; “A Quantum Physics Search for Liver-Stage Antimalarials, Grand Challenges Explorations Grant,” Bill & Melinda Gates Foundation, 2012; “Identification of Nrf2 Activators Using an In Silico Modeling Platform, Followed by Evaluation of These Compounds in an Alpha-Synuclein Model of PD,” MJFF Research Grant, Michael J. Fox Foundation for Parkinson's Research, 2011; David J. Sullivan, Yi Liu, Bryan T. Mott, Nikola Kaludov and Martin N. Martinov. “Discovery of Novel Liver-Stage Antimalarials Through Quantum Similarity,” PLOS ONE, May 7, 2015, DOI: 10.1371/journal.pone.0125593; T.P. Williamson, S. Amirahmadi, G.Joshi, N.K.Kaludov, M. N. Martinov, D. A. Johnson and J. A. Johnson “Discovery of Potent, Novel Nrf2 Inducers via Quantum Modeling, Virtual Screening, and In Vitro Experimental Validation,” Chem. Biol. Drug Des. 6:810, 2012; D. J. Sullivan Jr, N. Kaludov and M. N. Martinov “Discovery of Potent, Novel, Non-toxic Anti-malarial Compounds via Quantum Modeling, Virtual Screening and In Vitro Experimental Validation,” Malaria Journal, 10:274, 2011; Hopkins A L. “Network pharmacology,” Nat Biotechnol. 2007 October; 25 (10):1110-1; Vidal M, Cusick M E, Barabási AL. “Interactome networks and human disease,” Cell 2011 Mar. 18; 144 (6): 986-98; Thomas J P, Modos D, Korcsmaros T and Brooks-Warburton J (2021) “Network Biology Approaches to Achieve Precision Medicine in Inflammatory Bowel Disease,” Front. Genet. 12:760501; Ma C, Xu T, Sun X, Zhang S, Liu S, Fan S, Lei C, Tang F, Zhai C, Li C, Luo J, Wang Q, Wei W, Wang X, Cheng F. “Network Pharmacology and Bioinformatics Approach Reveals the Therapeutic Mechanism of Action of Baicalein in Hepatocellular Carcinoma,” Evid Based Complement Alternat Med. 2019 Feb. 12 2019:7518374; Zheng S, Xue T, Wang B, Guo H and Liu Q (2022), “Application of network pharmacology in the study of the mechanism of action of traditional Chinese medicine in the treatment of COVID-19, ” Front. Pharmacol. 13:926901; Tebani A, Afonso C, Bekri S. “Advances in metabolome information retrieval: turning chemistry into biology. Part I: analytical chemistry of the metabolome,” J Inherit Metab Dis. 2018 May; 41 (3):379-391. doi:10.1007/s10545-017-0074-y. Epub 2017 Aug. 24, each of which is incorporated by reference in its entirety.

### Example 4

### Chromatographic System and ML-Algorithm Overview

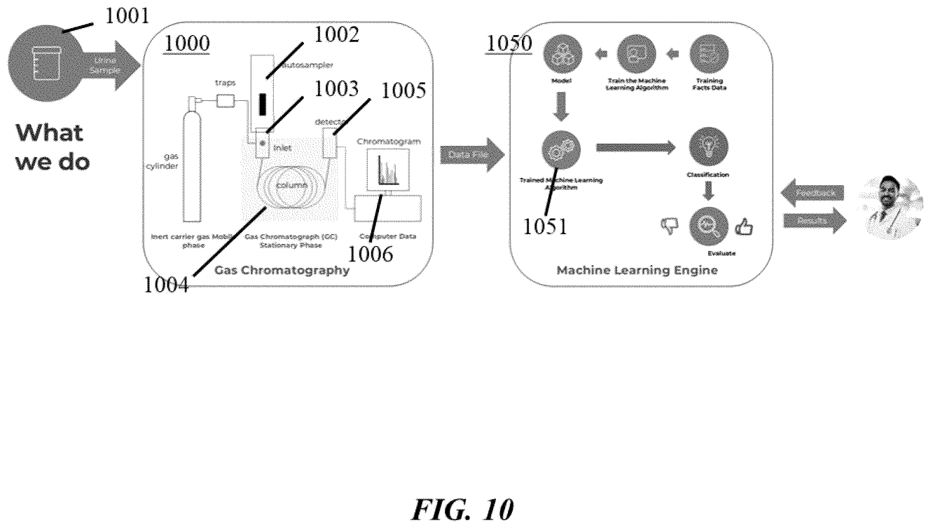

FIG. 10 shows a schematic of a chromatographic system 1000 configured to input data into an ML-algorithm 1050. A urine sample 1001 can be processed as described elsewhere herein (e.g., unprocessed, derivativized, etc.) and injected via an autosampler 1002 into an inlet 1003 of a gas chromatography system. An inert carrier gas can be flowed through chromatography column 1004 as a temperature of the inlet 1003 is increased at a predetermined rate, and detector 1005 can detect passage of components of the sample 1001 and provide that information as chromatographic data 1006. The chromatographic data can then be input into a trained ML algorithm 1051, which can provide a classification of one or more disease states. The classification can be evaluated for the presence or absence of the disease state and communicated to a user (e.g., physician). Alternatively, the classification can be provided directly to the user without evaluation. The user can provide feedback to the ML-algorithm to, for example, improve the algorithm, improve the classification, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for determining a metabolic profile of a subject, comprising:
(a) obtaining chromatography data obtained from a biological sample of said subject;
(b) processing, using a machine-learning (ML) algorithm, a set of input features of said chromatography data to generate output data, wherein said set of input features do not comprise a presence or a quantity of a metabolite of said biological sample; and
(c) determining said metabolic profile based at least in part on said output data.

2. The method of claim 1, further comprising, (d) processing said metabolic profile to determine a presence or an absence of a disease state.

3. The method of claim 2, wherein said processing in (d) further comprises processing a characteristic of said subject.

4. The method of claim 3, wherein said characteristic is selected from the group consisting of previous tumor, demographics, clinical characteristic, demographic characteristic, and phenotypic characteristic.

5. The method of claim 2, wherein said disease state is selected from the group consisting of an oncological disease, an infectious disease, a chronic disease, a nutritional deficiency, an environmental disease, an autoimmune disorder, and a genetic disease.

6. The method of claim 2, wherein said disease state comprises a plurality of disease states, and wherein the method further comprises processing the metabolic profile to determine a presence or absence of each of the plurality of disease states.

7. The method of claim 6, wherein said plurality of disease states comprises at least about 5 disease states.

8. The method of claim 7, wherein said plurality of disease states comprises at least about 50 disease states.

9. The method of claim 2, wherein said presence or said absence of said disease state is determined at an accuracy of at least about 85%.

10. The method of claim 2, wherein said presence or said absence of said disease state is determined using a single sample from said subject.

11. The method of claim 1, further comprising processing said metabolic profile to determine a presence or an absence of use of a compound by said subject.

12. The method of claim 1, wherein said biological sample is a urine sample.

13. The method of claim 1, wherein said chromatography data is derived from a gas chromatography system.

14. The method of claim 1, wherein said chromatography data is derived from a liquid chromatography system.

15. The method of claim 1, wherein said set of input features do not comprise a presence or a quantity of an analyte of said biological sample.

16. The method of claim 1, wherein said ML algorithm comprises a fuzzy decision network.

17. The method of claim 1, further comprising repeating (a)-(c) for a plurality of biological samples of a plurality of subjects to generate a plurality of metabolic profiles.

18. The method of claim 17, further comprising analyzing said plurality of metabolic profiles to determine a differential feature of said plurality of metabolic profiles.

19. The method of claim 1, further comprising, prior to (a), performing chromatography on said biological sample to generate said chromatography data.

20. The method of claim 19, wherein said chromatography does not comprise derivatization of said biological sample.

21. The method of claim 1, wherein said biological sample is an unpreserved biological sample.

22. The method of claim 21, wherein said unpreserved biological sample is a raw biological sample.

23. The method of claim 1, further comprising, subsequent to (a), processing said sample with a mass spectrometer.

24. The method of claim 1, further comprising, determining said presence or said quantity of said metabolite.

25. The method of claim 24, further comprising determining said metabolic profile based further on said presence or said quantity of said metabolite.

26. The method of claim 1, wherein said output data comprises said presence or said quantity of said metabolite.

27. The method of claim 1, wherein said chromatography data comprises a first gas chromatography data and a second liquid chromatography data.

28. The method of claim 1, wherein said set of input features further comprises additional data.

29. The method of claim 28, wherein said additional data comprises additional data selected from the group consisting of additional chromatography data and additional optical data.

30. A method of determining a multi-omic profile of a subject, comprising:
(a) obtaining chromatography data obtained from a biological sample of said subject;
(b) processing, using a machine-learning (ML) algorithm, a set of input features of said chromatography data to generate output data, wherein said set of input features do not comprise a presence or a quantity of a metabolite of said biological sample; and
(c) determining said multi-omic profile based at least in part on said output data.

31. The method of claim 30, further comprising, (d) processing said metabolic profile to determine a presence or an absence of a disease state.

32. The method of claim 31, wherein said processing in (d) further comprises processing a characteristic of said subject.

33. The method of claim 32, wherein said characteristic is selected from the group consisting of previous tumor, demographics, clinical characteristic, demographic characteristic, and phenotypic characteristic.

34. The method of claim 31, wherein said disease state is selected from the group consisting of an oncological disease, an infectious disease, a chronic disease, a nutritional deficiency, an environmental disease, an autoimmune disorder, and a genetic disease.

35. The method of claim 31, wherein said disease state comprises a plurality of disease states, and wherein the method further comprises processing the metabolic profile to determine a presence or absence of each of the plurality of disease states.

36. The method of claim 35, wherein said plurality of disease states comprises at least about 5 disease states.

37. The method of claim 36, wherein said plurality of disease states comprises at least about 50 disease states.

38. The method of claim 31, wherein said presence or said absence of said disease state is determined at an accuracy of at least about 85%.

39. The method of claim 31, wherein said presence or said absence of said disease state is determined using a single sample from said subject.

40. The method of claim 30, further comprising processing said metabolic profile to determine a presence or an absence of use of a compound by said subject.

41. The method of claim 30, wherein said biological sample is a urine sample.

42. The method of claim 30, wherein said chromatography data is derived from a gas chromatography system.

43. The method of claim 30, wherein said chromatography data is derived from a liquid chromatography system.

44. The method of claim 30, wherein said set of input features do not comprise a presence or a quantity of an analyte of said biological sample.

45. The method of claim 30, wherein said ML algorithm comprises a fuzzy decision network.

46. The method of claim 30, further comprising repeating (a)-(c) for a plurality of biological samples of a plurality of subjects to generate a plurality of metabolic profiles.

47. The method of claim 46, further comprising analyzing said plurality of metabolic profiles to determine a differential feature of said plurality of metabolic profiles.

48. The method of claim 30, further comprising, prior to (a), performing chromatography on said biological sample to generate said chromatography data.

49. The method of claim 48, wherein said chromatography does not comprise derivatization of said biological sample.

50. The method of claim 30, wherein said biological sample is an unpreserved biological sample.

51. The method of claim 50, wherein said unpreserved biological sample is a raw biological sample.

52. The method of claim 30, further comprising, subsequent to (a), processing said sample with a mass spectrometer.

53. The method of claim 30, further comprising, determining said presence or said quantity of said metabolite.

54. The method of claim 53, further comprising determining said metabolic profile based further on said presence or said quantity of said metabolite.

55. The method of claim 30, wherein said output data comprises said presence or said quantity of said metabolite.

56. The method of claim 30, wherein said chromatography data comprises a first gas chromatography data and a second liquid chromatography data.

57. The method of claim 30, wherein said set of input features further comprises additional data.

58. The method of claim 57, wherein said additional data comprises additional data selected from the group consisting of additional chromatography data and additional optical data.

59. A system, comprising:
a cartridge configured to receive a biological sample from a subject; and
a chromatography system configured to receive said cartridge and process at least a portion of said biological sample.

60. The system of claim 59, wherein said cartridge comprises a test strip.

61. The system of claim 59, wherein the cartridge comprises a collection cup.

62. The system of claim 59, wherein said chromatography system comprises a gas chromatography system.

63. The system of claim 59, wherein said chromatography system comprises a liquid chromatography system.

64. The system of claim 59, wherein said chromatography system is a single use chromatography system.

65. The system of claim 59, further comprising a data connection configured to transmit data associated with said at least a portion of said biological sample from said chromatography system.

66. The system of claim 65, wherein said data connection comprises a wireless data connection.

67. The system of claim 59, further comprising an autosampler configured to provide a plurality of cartridges comprising said cartridge to said chromatography system.

68. The system of claim 59, wherein said chromatography system comprises one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), and conductivity detector.

69. A system, comprising:
a chromatography system configured to generate chromatography data comprising a set of input features from at least a portion of a biological sample of a subject;
one or more computer processors operatively coupled to said chromatography system, wherein said one or more computer processors are individually or collectively programmed to process, using a machine-learning (ML) algorithm, said chromatography data to generate output data related to a metabolic profile of said subject.

70. The system of claim 69, wherein said cartridge comprises a test strip.

71. The system of claim 69, wherein the cartridge comprises a collection cup.

72. The system of claim 69, wherein said chromatography system comprises a gas chromatography system.

73. The system of claim 69, wherein said chromatography system comprises a liquid chromatography system.

74. The system of claim 69, wherein said chromatography system is a single use chromatography system.

75. The system of claim 69, further comprising a data connection configured to transmit data associated with said at least a portion of said biological sample from said chromatography system to the one or more computer processors.

76. The system of claim 75, wherein said data connection comprises a wireless data connection.

77. The system of claim 69, further comprising an autosampler configured to provide a plurality of cartridges comprising said cartridge to said chromatography system.

78. The system of claim 69, wherein said chromatography system comprises one or more of a flame ionization detector (FID), thermal conductivity detector (TCD), electron capture detector (ECD), photoionization detector (PID), mass spectrometer (MS), ion mobility spectrometer (IMS), nitrogen-phosphorus detector (NPD), Raman detector, ultraviolet-visible (UV-Vis) detector, photodiode array detector (PDA), fluorescence detector, evaporative light scattering detector (ELSD), refractive index detector (RID), and conductivity detector.
